(19) United States
(12) Patent Application Publication
(10) Pub. No.: US 2021/0054050 A1
(43) Pub. Date: Feb. 25, 2021

(54) FORMULATION OPTIMIZATION FOR BISPECIFIC ANTIBODIES

(71) Applicant: Regeneron Pharmaceuticals, Inc., Tarrytown, NY (US)

(72) Inventors: Chen Zhou, Cortlandt Manor, NY (US); Wenhua Wang, Eastchester, NY (US); Dingjiang Liu, Pleasantville, NY (US)

(21) Appl. No.: 16/998,391

(22) Filed: Aug. 20, 2020

Related U.S. Application Data
(60) Provisional application No. 62/889,354, filed on Aug. 20, 2019.

(51) Int. Cl. C07K 16/00; G16B 15/00

(52) U.S. Cl. CPC C07K 16/00; G16B 15/00; C07K 2317/92; C07K 2317/94; C07K 2317/31

(57) ABSTRACT

The present invention provides methods and systems for formulation optimization of bispecific antibodies. The present application also provides methods and systems to select molecule candidates for constructing bispecific antibodies and formulation optimization thereof. Physico-chemical parameters of a bispecific antibody are characterized. The formulation optimization strategies are guided by the prediction of interaction parameters. Various formulation optimization strategies are provided based on these physico-chemical parameters.

### CROSS-REFERENCE TO RELATED APPLICATIONS

This application claims priority to and the benefit of U.S. Provisional Patent Application No. 62/889,354, filed on Aug. 20, 2019 (20. 08. 2019), the content of which is incorporated herein by reference in its entirety.

### FIELD

The present invention generally pertains to methods and systems for formulation optimization of bispecific antibodies. The present invention also provides methods and systems for selecting a combination of peptides or proteins to generate bispecific antibodies and provides methods for formulation optimization thereof.

### BACKGROUND

Bispecific antibodies are highly valuable biopharmaceutical products with enhanced efficacy and target specificity in comparison to conventional monoclonal antibodies, since bispecific antibodies target two different antigens. The designs of bispecific antibodies can be directed to multiple tissue-specific antibodies combined with small molecule drugs, such as combining multiple tissue-specific antibodies and cytotoxic drugs to release drugs in close proximity to tumors. Small drug molecules can be conjugated to the purified bispecific antibodies to produce antibody-drug conjugates (ADC). However, drug development and formulation optimization of bispecific antibodies can be challenging due to their structure and composition complexity, since the two Fab arms of bispecific antibodies are heterogeneous, derived from two different parental antibodies.

The two heterogeneous Fab arms of bispecific antibodies can have different physico-chemical properties, such as differences in surface hydrophobicity or surface charges. The structural complexity of bispecific antibodies leads to changes in physico-chemical properties which have negative or adverse impacts on aqueous solubility. The challenges include reduced solubility due to high viscosity or opalescence during formulation development of bispecific antibodies. Aqueous solubility is a constraint to bioavailability of drug formulations. The development of stable protein-based formulation is critical for safety issues relevant to immunogenic response, drug stability during reasonable shelf life, and delivery optimization through injection. It is beneficial to understand protein stability and solubility under various formulation conditions, such as pH, ionic strength, buffer salts, or temperature, for optimizing formulations of bispecific antibodies.

It will be appreciated that a need exists for methods and systems to select a combination of peptides or proteins with target physico-chemical properties for producing bispecific antibodies. A need further exists for methods to optimize compositions for formulation development of bispecific antibodies.

### SUMMARY

The structural complexity of bispecific antibodies due to heterogeneous Fab arms can lead to negative or adverse impacts on aqueous solubility. Challenges include high viscosity or opalescence during formulation development of bispecific antibodies. The present application provides methods and systems to select molecule candidates for constructing bispecific antibodies and formulation optimization thereof. A profile of physico-chemical parameters of a bispecific antibody and its parental antibodies are characterized. Various formulation optimization strategies are provided based on these physico-chemical parameters.

The disclosure provides a method for producing a combination of peptides or proteins with target physico-chemical properties, comprising: receiving a plurality of amino acid sequences of the peptides or proteins; selecting peptides or proteins having desired amino acid sequences; determining a profile of protein-protein interactions of the peptides or proteins having desired amino acid sequences; selecting a target profile of the protein-protein interactions of the peptides or proteins having desired amino acid sequences; and producing a combination of peptides or proteins having desired amino acid sequences according to the target profile of the protein-protein interactions. In some exemplary embodiments, in the method of the present application, the protein-protein interactions can be repulsive or attractive protein-protein interactions, wherein the profile of the protein-protein interactions can be determined by measuring interaction parameters of the peptides or proteins having desired amino acid sequences.

In some exemplary embodiments, the method of the present application further comprises determining a profile of physico-chemical properties of the peptides or proteins having desired amino acid sequences, wherein the combination of peptides or proteins having desired amino acid sequences can be produced according to the target profile of the protein-protein interaction and the profile of the physico-chemical properties, wherein the physico-chemical property can be a theoretical isoelectric point, an experimental isoelectric point, surface hydrophobicity, relative surface hydrophobicity, a hydrophobicity index, surface charges, charge heterogeneity, second osmotic vial coefficient, agitation stability, opalescence, viscosity, or interfacial sensitivity. In some exemplary embodiments, the surface hydrophobicity or surface charges can be determined by conducting a structural modeling of the peptides or proteins having desired amino acid sequences.

In some preferred exemplary embodiments, in the method of the present application, a concentration of the combination of the peptides or proteins having desired amino acid sequences can be from about 20 mg/mL to about 200 mg/mL, or at least about 70 mg/mL, or at least about 100 mg/mL.

In some exemplary embodiments, in the method of the present application, the combination of the peptides or proteins having desired amino acid sequences can be a bispecific antibody or a multi-specific antibody, wherein the method of the present application further comprises determining a hydrophobicity index, surface charges, or charge heterogeneity of a variable region of the bispecific antibody or the multi-specific antibody for producing the bispecific antibody or the multi-specific antibody.

The disclosure, at least in part, provides a system for producing a combination of peptides or proteins with target physico-chemical properties, comprising: a plurality of amino acid sequences of the peptides or proteins; a selection of the peptides or proteins having desired amino acid sequences; a profile of protein-protein interactions of the peptides or proteins having desired amino acid sequences; a target profile of the protein-protein interactions of the peptides or proteins having desired amino acid sequences; and a combination of peptides or proteins having desired amino acid sequences, wherein the peptides or proteins having desired amino acid sequences are selected according to the target profile of the protein-protein interactions. In some exemplary embodiments, in the system of the present application, the protein-protein interactions can be repulsive or attractive protein-protein interactions, wherein the profile of the protein-protein interactions is determined by measuring interaction parameters of the peptides or proteins having desired amino acid sequences.

In some exemplary embodiments, the system of the present application further comprises a profile of physico-chemical properties of the peptides or proteins having desired amino acid sequences, wherein the combination of peptides or proteins having desired amino acid sequences is selected according to the target profile of the protein-protein interaction and the profile of the physico-chemical properties, wherein the physico-chemical property can be a theoretical isoelectric point, an experimental isoelectric point, surface hydrophobicity, relative surface hydrophobicity, a hydrophobicity index, surface charges, charge heterogeneity, second osmotic vial coefficient, agitation stability, opalescence, viscosity, or interfacial sensitivity. In some exemplary embodiments, the surface hydrophobicity or surface charges can be determined by conducting a structural modeling of the peptides or proteins having desired amino acid sequences.

In some preferred exemplary embodiments, in the system of the present application, a concentration of the combination of the peptides or proteins having desired amino acid sequences can be from about 20 mg/mL to about 200 mg/mL, or at least about 70 mg/mL, or at least about 100 mg/mL.

In some exemplary embodiments, in the system of the present application, the combination of the peptides or proteins having desired amino acid sequences can be a bispecific antibody or a multi-specific antibody, wherein the system of the present application further comprises a profile of a hydrophobicity index, surface charges, or charge heterogeneity of a variable region of the bispecific antibody or the multi-specific antibody.

The disclosure, at least in part, provides a method for optimizing or selecting at least one component in a formulation, wherein the formulation comprises the combination of the peptides or proteins having desired amino acid sequences of the present application, the method comprising: adjusting ionic strength of the formulation based on the target profile of the protein-protein interactions of the peptides or proteins having desired amino acid sequences, and adjusting a pH value of the formulation based on the target profile of the protein-protein interactions of the peptides or proteins having desired amino acid sequences.

In some preferred exemplary embodiments, the method of formulation optimization in present application further comprises adding a salt to the formulation based on the target profile of the protein-protein interactions of the peptides or proteins having desired amino acid sequences. In some preferred exemplary embodiments, the method of formulation optimization in present application further comprises adding a hydrophobic excipient to the formulation based on the target profile of the protein-protein interactions of the peptides or proteins having desired amino acid sequences, wherein the at least one component is sodium chloride, acetate, histidine, or arginine hydrochloride.

The disclosure, at least in part, provides a method of optimizing formulation of bispecific or multi-specific antibodies including a method for optimizing or selecting at least one component in a formulation, wherein the formulation comprises a bispecific antibody or a multi-specific antibody, the method comprising: determining a profile of protein-protein interactions of the bispecific antibody or the multi-specific antibody; and optimizing or selecting the at least one component in the formulation based on the profile of the protein-protein interactions of the bispecific antibody or the multi-specific antibody.

In some exemplary embodiments, in the method of optimizing formulation of bispecific or multi-specific antibody, the profile of the protein-protein interactions can be determined by measuring interaction parameters of the bispecific antibody or the multi-specific antibody. In some exemplary embodiments, the method of optimizing formulation of bispecific or multi-specific antibody further comprises adjusting ionic strength of the formulation based on the profile of the protein-protein interactions of the bispecific antibody or the multi-specific antibody, or adjusting a pH value of the formulation based on the profile of the protein-protein interactions of the bispecific antibody or the multi-specific antibody.

In some exemplary embodiments, the method of optimizing formulation of bispecific or multi-specific antibody further comprises determining a profile of physico-chemical properties of the bispecific antibody or the multi-specific antibody, wherein optimizing or selecting the at least one component in the formulation is based on the profile of the protein-protein interactions of the bispecific antibody or the multi-specific antibody and the profile of the physico-chemical properties of the bispecific antibody or the multi-specific antibody.

In some exemplary embodiments, the method of optimizing formulation of bispecific or multi-specific antibody further comprises adding a salt to the formulation based on the profile of protein-protein interactions of the bispecific antibody or the multi-specific antibody, or adding a hydrophobic excipient to the formulation based on the profile of protein-protein interactions of the bispecific antibody or the multi-specific antibody. In some exemplary embodiments, in the method of optimizing formulation of bispecific or multi-specific antibody, the at least one component is sodium chloride, acetate, histidine, or arginine hydrochloride.

In some exemplary embodiments, in the method of optimizing formulation of bispecific or multi-specific antibody, the physico-chemical property is a theoretical isoelectric point, an experimental isoelectric point, surface hydrophobicity, relative surface hydrophobicity, a hydrophobicity index, surface charges, charge heterogeneity, second osmotic vial coefficient, agitation stability, opalescence, viscosity, or interfacial sensitivity, wherein the surface hydrophobicity or surface charges can be determined by conducting a structural modeling of the bispecific antibody or the multi-specific antibody.

In some exemplary embodiments, in the method of optimizing formulation of bispecific or multi-specific antibodies, the protein-protein interactions are repulsive or attractive protein-protein interactions. In some exemplary embodiments, in the method of optimizing formulation of bispecific or multi-specific antibodies, a concentration of the bispecific antibody or the multi-specific antibody is from about 20 mg/mL to about 200 mg/mL, or at least about 70 mg/mL, or at least about 100 mg/mL. In some exemplary embodiments, the method of optimizing formulation of bispecific or multi-specific antibody further comprises determining a hydrophobicity index, surface charges, or charge heterogeneity of a variable region of the bispecific antibody or the multi-specific antibody.

In some exemplary embodiments, the method of optimizing formulation of bispecific or multi-specific antibody further comprises determining a profile of physico-chemical properties of the bispecific antibody or the multi-specific antibody, wherein optimizing or selecting the at least one component in the formulation is based on the profile of the protein-protein interactions of the bispecific antibody or the multi-specific antibody and the profile of the physico-chemical properties of the bispecific antibody or the multi-specific antibody.

In some exemplary embodiments, the method of optimizing formulation of bispecific or multi-specific antibody further comprises adding a salt to the formulation based on the profile of protein-protein interactions of the bispecific antibody or the multi-specific antibody, or adding a hydrophobic excipient to the formulation based on the profile of protein-protein interactions of the bispecific antibody or the multi-specific antibody. In some exemplary embodiments, in the method of optimizing formulation of bispecific or multi-specific antibody, the at least one component is sodium chloride, acetate, histidine, or arginine hydrochloride.

In some exemplary embodiments, in the method of optimizing formulation of bispecific or multi-specific antibody, the physico-chemical property is a theoretical isoelectric point, an experimental isoelectric point, surface hydrophobicity, relative surface hydrophobicity, a hydrophobicity index, surface charges, charge heterogeneity, second osmotic vial coefficient, agitation stability, opalescence, viscosity, or interfacial sensitivity, wherein the surface hydrophobicity or surface charges is determined by conducting a structural modeling of the bispecific antibody or the multi-specific antibody.

In some exemplary embodiments, in the method of optimizing formulation of bispecific or multi-specific antibody, the protein-protein interactions are repulsive or attractive protein-protein interactions. In some exemplary embodiments, in the method of optimizing formulation of bispecific or multi-specific antibody, a concentration of the bispecific antibody or the multi-specific antibody is from about 20 mg/mL to about 200 mg/mL, or at least about 70 mg/mL, or at least about 100 mg/mL. In some exemplary embodiments, the method of optimizing formulation of bispecific or multi-specific antibody further comprises determining a hydrophobicity index, surface charges, or charge heterogeneity of a variable region of the bispecific antibody or the multi-specific antibody.

These, and other, aspects of the invention will be better appreciated and understood when considered in conjunction with the following description and the accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1A shows a surface map of Fab of mAb-B based on structural modeling according to an exemplary embodiment. FIG. 1B shows a surface map of Fab of mAb-A based on structural modeling according to an exemplary embodiment. FIG. 1C shows a surface map of BsAb1 based on structural modeling according to an exemplary embodiment. The shaded areas in the rectangles indicate the locations of hydrophobic patches. The shaded areas in the circles indicate the locations of negative charge patches. The shaded areas in the triangles indicate positive charge patches.

FIGS. 2A-2C show measurements of optical densities at OD 405 nm of BsAb1, mAb-A, and mAb-B formulations to characterize opalescence of protein formulations according to an exemplary embodiment. A5 indicates the buffer composition of 10 mM acetate at pH 5. H6 indicates the buffer composition of 10 mM histidine at pH 6. H6N indicates the buffer composition of 10 mM histidine, 150 mM NaCl at pH 6.

FIGS. 3A-3C show measurements of viscosities for BsAb1, mAb-A, and mAb-B formulations according to an exemplary embodiment. Theoretical viscosity of immunoglobulin with 10 nm diameter at 150 mg/mL was calculated by Mooney equation as a comparison. A5 indicates the buffer composition of 10 mM acetate at pH 5. H6 indicates the buffer composition of 10 mM histidine at pH 6. H6N indicates the buffer composition of 10 mM histidine, 150 mM NaCl at pH 6. H6Arg indicates the buffer composition of 10 mM histidine, 150 mM ArgHCl at pH 6.

FIG. 4 shows the measurements of agitation stabilities to investigate the interfacial sensitivity of BsAb1 in various formulations according to an exemplary embodiment. The measurements include control and agitated protein formulations. H6 indicates the buffer composition of 10 mM histidine at pH 6. H6N indicates the buffer composition of 10 mM histidine, 150 mM NaCl at pH 6. H6Arg indicates the buffer composition of 10 mM histidine, 150 mM ArgHCl at pH 6.

FIG. 5A shows the measurements of interaction parameters (k_{D}) for BsAb1, mAb-A, and mAb-B in various buffer compositions, including co-formulations of mAb-A and mAb-B, according to an exemplary embodiment. A5 indicates the buffer composition of 10 mM acetate at pH 5. H6 indicates the buffer composition of 10 mM histidine at pH 6. H6N indicates the buffer composition of 10 mM histidine, 150 mM NaCl at pH 6. H6Arg indicates the buffer composition of 10 mM histidine, 150 mM ArgHCl at pH 6. FIG. 5B shows the measurements of second osmotic viral coefficient B₂₂ of BsAb1 formulation in 10 mM histidine at pH 6 at various protein concentrations using composition gradient-multi-angle light scattering (CG-MALS) according to an exemplary embodiment.

FIG. 6A shows correlation analysis for opalescence and interaction parameter k_{D} according to an exemplary embodiment (at a concentration of 150 mg/mL). FIG. 6B shows correlation analysis for viscosity and interaction parameter k_{D} according to an exemplary embodiment. FIG. 6C shows correlation analysis for opalescence and interaction parameter k_{D} according to an exemplary embodiment (at a concentration of 70 mg/mL).

### DETAILED DESCRIPTION

Bispecific antibodies are next-generation antibodies aiming for superior therapeutic effects with two different antigen-binding sites which can enhance efficacy and target specificity in comparison to conventional monoclonal antibodies. The application of bispecific antibodies spans a wide range of therapeutic areas including autoimmune, oncology, or chronic inflammatory indications. For example, in cancer therapy, bispecific antibodies can achieve stimulation of various immune-receptors simultaneously to trigger and enhance tumor cytotoxic immune response.

The administration of bispecific antibodies is dominantly parenteral, such as intravenous or subcutaneous injection. The demand of high protein concentration formulations is increasing due to the requirement of small injection volume in subcutaneous dosage to improve patient compliance. Commonly, the demand for protein concentration may be targeting above 100 mg/mL in a subcutaneous formulation. However, the development of protein formulation with high concentrations can be challenging, since protein molecules tend to aggregate and/or precipitate at high concentrations that may lead to high viscosity and opalescence. Proteins generally have higher tendency of self-association at high concentrations.

The two Fab arms of bispecific antibodies are heterogeneous, since they derive from two different parental antibodies. The structure and composition complexity of bispecific antibodies can cause challenges in formulation development, such as the issues of opalescence, high viscosity, or interfacial sensitivity, since the two heterogeneous Fab arms can have significantly different physico-chemical properties. The present application provides a method and system to select molecule candidates for constructing bispecific antibodies, such as selecting a combination of peptides or proteins to produce bispecific antibodies based on the profile of the protein-protein interaction and/or the profile of the physico-chemical properties of the peptides or proteins having desired amino acid sequences.

The present application further provides a method for formulation optimization of the bispecific antibody which can be produced using the methods of the present application. The present application provides methods and systems to investigate the molecular mechanism of the adverse protein behaviors of bispecific antibodies at high concentration by structural modeling of the bispecific antibody and its parental antibodies. A profile of physico-chemical parameters can be characterized and compared among the bispecific antibody and its parental antibodies. Various formulation optimization strategies are provided based on the physico-chemical parameters.

The present application provides methods and systems to predict protein behavior using protein interaction parameter k_{D} to quantify protein-protein interactions. In particular, the present application provides characterizations of the molecular mechanism of bispecific antibodies in solution, in particular at high protein concentrations. The present application provides methods and systems to explore the impacts of various formulation conditions, such as adjusting ionic strength, pH value, or buffer salts, to reduce the opalescence and high viscosity during formulation development for bispecific antibodies.

Protein-protein interactions, such as repulsive or attractive protein-protein interactions, are relevant to protein behaviors at high protein concentrations in solution. Repulsive protein-protein interactions are generally preferred, since the attractive protein-protein interactions may attribute to adverse protein behaviors. Methods for characterizing protein-protein interactions include dynamic light scattering (DLS), static light scattering (SLS), small angle X-rays (SAXS), analytical ultracentrifugation (AUC) and membrane osmometry. In DLS measurement, the nature and magnitude of protein-protein interactions can be extrapolated as interaction parameter k_{D} from the non-ideal dependence of diffusion coefficient on protein concentration in the relatively diluted regime. SLS measures the non-ideal light scattering intensity change upon protein concentration gradient. Second osmotic viral coefficient B₂₂ can be measured using SLS.

Protein-protein interactions can dominate the characteristics of proteins (protein behavior) at high protein concentration in solution. The present application provides a method and system to predict the behaviors of bispecific antibodies at high protein concentration using interaction parameter k_{D}. Interaction parameter k_{D} can provide reasonable prediction for protein high concentration behaviors for selecting molecule candidates to constructing bispecific antibodies and formulation optimization thereof. The method and system of the present application can be used to select candidate molecules to generate bispecific antibodies by measuring k_{D} to predict protein behaviors. In addition, the present application provides a method for optimizing or selecting at least one component in a formulation containing bispecific antibodies.

The method of the present application provides prediction to obtain reasonable correlation between opalescence/viscosity and interaction parameter k_{D} for formulation optimization of bispecific antibodies. The method of the present application also provides prediction to obtain reasonable correlation between opalescence/viscosity and protein-protein interactions for formulation optimization of bispecific antibodies.

In some exemplary embodiments, the physico-chemical properties of protein-protein interactions in bispecific antibody can be predominately electrostatic, and the strategies of increasing ionic strength and adjusting pH value can effectively improve the outcome of formulation optimization for bispecific antibodies.

The present application provides a method to optimize formulations containing a bispecific antibody, wherein the method comprises determining a profile of protein-protein interactions of the bispecific antibody, such as the attractive protein-protein interactions. The opalescence and/or viscosity of the bispecific antibody formations can be significantly reduced by adjusting the ionic strength or pH value of the formulation based on the profile of the protein-protein interactions of the bispecific antibody, such as by increasing ionic strength or reducing pH value to reduce opalescence and viscosity through mitigating attractive protein-protein interactions.

The superior therapeutic effects of bispecific antibodies have led to an increasing demand for formulation optimization of the bispecific antibodies. Exemplary embodiments disclosed herein satisfy the aforementioned demands by providing methods and systems to satisfy the aforementioned demands by providing methods and systems to select a combination of peptides or proteins to produce bispecific antibodies based on target profile of the protein-protein interactions of the peptides or proteins having desired amino acid sequences according to the measurement of interaction parameter k_{D}. This disclosure also provides a method for optimizing formulations containing bispecific antibodies. The optimization strategies can be guided by the prediction of interaction parameter k_{D}. These strategies also address long felt needs of solving the problems of high viscosity or opalescence during formulation development of bispecific antibodies.

The term “a” should be understood to mean “at least one”; and the terms “about” and “approximately” should be understood to permit standard variation as would be understood by those of ordinary skill in the art; and where ranges are provided, endpoints are included.

As used herein, the terms “include,” “includes,” and “including,” are meant to be non-limiting and are understood to mean “comprise,” “comprises,” and “comprising,” respectively.

In some exemplary embodiments, the disclosure provides a method for producing a combination of peptides or proteins with target physico-chemical properties, comprising: receiving a plurality of amino acid sequences of the peptides or proteins; selecting the peptides or proteins having desired amino acid sequences, determining a profile of protein-protein interactions of the peptides or proteins having desired amino acid sequences; selecting a target profile of the protein-protein interactions of the peptides or proteins having desired amino acid sequences; and producing a combination of peptides or proteins having desired amino acid sequences according to the target profile of the protein-protein interactions.

As used herein, the term “peptides” or “proteins” includes any amino acid polymer having covalently linked amide bonds. Proteins comprise one or more amino acid polymer chains, generally known in the art as “peptide” or “polypeptides.” A protein may contain one or multiple polypeptides to form a single functioning biomolecule. In some exemplary embodiments, the protein can be an antibody, a bispecific antibody, a multi-specific antibody, antibody fragment, monoclonal antibody, host-cell protein or combinations thereof.

In some exemplary embodiments, the disclosure provides a method for optimizing or selecting at least one component in a formulation, wherein the formulation comprises a bispecific antibody or a multi-specific antibody, the method comprising: determining a profile of protein-protein interactions of the bispecific antibody or the multi-specific antibody, and optimizing or selecting the at least one component in the formulation based on the profile of the protein-protein interactions of the bispecific antibody or the multi-specific antibody.

As used herein, an “antibody” is intended to refer to immunoglobulin molecules consisting of four polypeptide chains, two heavy (H) chains and two light (L) chains inter-connected by disulfide bonds. Each heavy chain has a heavy chain variable region (HCVR or VH) and a heavy chain constant region. The heavy chain constant region contains three domains, CH1, CH2 and CH3. Each light chain has of a light chain variable region and a light chain constant region. The light chain constant region consists of one domain (CL). The VH and VL regions can be further subdivided into regions of hypervariability, termed complementarity determining regions (CDR), interspersed with regions that are more conserved, termed framework regions (FR). Each VH and VL can be composed of three CDRs and four FRs, arranged from amino-terminus to carboxy-terminus in the following order: FR1, CDR1, FR2, CDR2, FR3, CDR3, FR4. The term “antibody” includes reference to both glycosylated and non-glycosylated immunoglobulins of any isotype or subclass. The term “antibody” is inclusive of, but not limited to, those that are prepared, expressed, created or isolated by recombinant means, such as antibodies isolated from a host cell transfected to express the antibody. An IgG comprises a subset of antibodies.

### Exemplary Embodiments

Embodiments disclosed herein provide methods and systems for producing a combination of peptides or proteins with target physico-chemical properties, comprising: receiving a plurality of amino acid sequences of the peptides or proteins; selecting the peptides or proteins having desired amino acid sequences, determining a profile of protein-protein interactions of the peptides or proteins having desired amino acid sequences; selecting a target profile of the protein-protein interactions of the peptides or proteins having desired amino acid sequences; and producing a combination of peptides or proteins having desired amino acid sequences according to the target profile of the protein-protein interactions.

In some exemplary embodiments, the method of this disclosure further comprises determining a profile of physico-chemical properties of the peptides or proteins having desired amino acid sequences, wherein the combination of peptides or proteins having desired amino acid sequences is produced according to the target profile of the protein-protein interaction and the profile of the physico-chemical properties. In some exemplary embodiments, the physico-chemical property is a theoretical isoelectric point, an experimental isoelectric point, surface hydrophobicity, relative surface hydrophobicity, a hydrophobicity index, surface charges, charge heterogeneity, second osmotic vial coefficient, agitation stability, opalescence, viscosity, or interfacial sensitivity.

In some preferred exemplary embodiments, in the method of the present application, a concentration of the combination of the peptides or proteins having desired amino acid sequences is from about 20 mg/mL to about 200 mg/mL, or at least about 70 mg/mL, or at least about 100 mg/mL, from about 1 mg/mL to about 400 mg/mL, from about 50 mg/mL to about 300 mg/mL, from about 100 mg/mL to about 300 mg/mL, from about 80 mg/mL to about 250 mg/mL, from about 80 mg/mL to about 150 mg/mL, at least about 50 mg/mL, at least about 67 mg/mL at least about 70 mg/mL, at least about 75 mg/mL, at least about 90 mg/mL, at least about 120 mg/mL, or at least about 150 mg/mL.

It is understood that the system is not limited to any of the aforesaid pharmaceutical products, peptides, proteins, antibodies, anti-drug antibodies, antigen-antibody complex, protein pharmaceutical products, chromatography column, or mass spectrometer.

The consecutive labeling of method steps as provided herein with numbers and/or letters is not meant to limit the method or any embodiments thereof to the particular indicated order

Various publications, including patents, patent applications, published patent applications, accession numbers, technical articles and scholarly articles are cited throughout the specification. Each of these cited references is incorporated by reference, in its entirety and for all purposes, herein. Unless described otherwise, all technical and scientific terms used herein have the same meaning as commonly understood by one of ordinary skill in the art to which this invention belongs.

The disclosure will be more fully understood by reference to the following Examples, which are provided to describe the disclosure in greater detail. They are intended to illustrate and should not be construed as limiting the scope of the disclosure.

### EXAMPLES

### Material and Reagent Preparation

### 1.1 Preparation of Bispecific Antibodies

Bispecific antibodies were prepared using “knob-in-hole” technique (Xu et al., Production of bispecific antibodies in “knobs-into-holes” using a cell-free expression system. mAbs, 2015, 7(1):231-242). BsAb1 is a IgG 4 monoclonal antibody which was constructed from parental mAb-A and mAb-B using “knob-in-hole” technique. BsAb1 has a common light chain and two different Fab arms. BsAb1 formulations showed high viscosity and opalescence at medium to high protein concentrations, such as a formulation containing 10 mM histidine at about pH 6 and about 70-85 mg/mL of BsAb1.

### 2.1 Preparation of Target Formulation Buffers

Various target formulation buffers were prepared as shown in Table 1, including the component of acetate, histidine, arginine hydrochloride, or sodium chloride in the range of about pH 5-8. All protein samples in their original formulation buffers were dialyzed into target formulation buffers, as shown in Table I. Protein concentrations were measured using variable path length UV/Vis spectrometer (Solo/VPE, C-Technologies Inc, NJ). All chemicals are reagent grade or higher.

TABLE 1
Compositions of target formulation buffers

| Target Formulation Buffer | Abbreviation |
|---|---|
| 10 mM acetate, pH 5 | A5 |
| 10 mM histidine, pH 6 | H6 |
| 10 mM histidine, 150 mM NaCl, pH 6 | H6N |
| 10 mM histidine, 150 mM ArgHCl, pH 6 | H6Arg |
| 10 mM Tris, pH 8 | T8 |

### Methods for Characterizing Physico-Chemical Properties of Antibodies

### 1.1 Turbidity Measurement

Optical density at 405 nm was measured with a UV/VIS auto scanner (Spectramax 190, Molecular Devices, CA) to quantify the turbidity and opalescence of protein formulations at room temperature.

### 2.1 Hydrophobic Interaction Chromatography-High Performance Liquid Chromatography (HIC-HPLC) for Obtaining Dimensionless Retention Time

Protein bound to t-butyl hydrophobic interaction chromatography (HIC) column (Tosoh Bioscience, PA) in Agilent 1200 HPLC instrument (Agilent, Santa Clara, Calif.) was eluted with decreasing gradient of (NH₄)₂SO₄ The retention time was obtained to calculate dimensionless retention time (DRT) using the below equation 1:

$$D R T = \frac{t s - t i}{t e - t i} \times 100 \% \qquad ( Equation 1 )$$

wherein tₛ is the elution time of the sample, tᵢ is the starting time of elution gradient, and t_{c} denotes the ending time of the gradient. DRT was used to rank the order of the relative surface hydrophobicity among protein molecules.

### 3.1 Microchip-Based Viscosity Measurement

VROC® Initium (Rheosense, CA) was used to measure the apparent viscosity of protein solutions at various formulation conditions. The temperature was set at 20° C. The viscosity reference standards of 2 cP and 80 cP were measured before and after the sample preparations or treatments to ensure the instrument performance. Intermediate shear rate was used to measure the apparent viscosity of protein solutions at various formulation conditions.

### 4.1 Imaged Capillary Isoelectric Focusing for Determination of Isoelectric Point

Imaged capillary isoelectric focusing (iCIEF), e.g. iCE3™ (Protein Simple, CA), was used to measure the isoelectric point (pI) of proteins. Protein pI was determined as the main peak in iCIEF measured charge profiles.

### 5.1 Homology Models of Proteins Including Sequence and Structural Analysis

The homology models of proteins were built with 5DWU framework using Molecular Operating Environment (MOE) (Chemical Computing Group, Quebec, Canada). The pI value was calculated based on the static model structure using the protein property analysis module. Surface properties were analyzed and calculated with BioMOE module based on algorithms from Sharma et al. (Sharma et al., In silico selection of therapeutic antibodies for development: Viscosity, clearance, and chemical stability. Proceedings of the National Academy of Sciences, 2014, 111(52): 18601-18606).

### 6.1 Measurements of Protein-Protein Interaction

Interaction parameter k_{D} and second osmotic viral coefficient B₂₂ were determined to measure protein-protein interaction. Interaction parameter k_{D} was measured using dynamic light scattering (DLS), such as Wyatt DynaPro plate reader (Wyatt Technology, CA), from 2 mg/mL to 10 mg/mL. The k_{D} value was extrapolated from the effect of macromolecule concentration on mutual diffusion coefficient, as shown in equation 2:

Dₘ=D₀(1+k_{D}c+ . . . ) (Equation 2)

wherein Dₘ is the mutual diffusion coefficient, Do is the value of Dₘ at infinite dilution, k_{D} is the first-order interaction parameter, c is protein concentration. At relatively diluted protein concentration range, the higher order concentration effect can be ignored and k_{D} equals to the slope divided by y-intercept in the linear plot of Dₘ vs c.

Second osmotic viral coefficient B₂₂ was measured using static light scattering (SLS), such as Wyatt composition gradient-multi-angle light scattering (CG-MALS) system (Calypso III coupled with a DAWN HELEOS MALS detector and an Optilab rEX refractive index detector, Wyatt Technology, CA). Protein solution at about 14 mg/mL was diluted in six steps to about 3 mg/mL in Calypso III with a flow rate of 1 mL/min. Light scattering intensity from multi-angles at 658 nm was used to determine the excess Rayleigh ratios and refractive index measurement was used to determine protein concentration. The following equations 3 and 4 previously described in the literature (Alford et al., High concentration formulations of recombinant human interleukin-1 receptor antagonist: I. Physical characterization. J Pharm Sci, 2008, 97(8):3035-3050; Kalonia et al., 2016. Effects of Protein Conformation, Apparent Solubility, and Protein-Protein Interactions on the Rates and Mechanisms of Aggregation for an IgG1 Monoclonal Antibody. The Journal of Physical Chemistry B, 2016, 120(29):7062-7075) were used to determine the second osmotic viral coefficient:

$$\frac{K c}{R ( θ )} = \frac{1}{{M}_{W}} + 2 {B}_{22} c \qquad ( Equation 3 )$$

wherein Mw is the mas-averaged molecular weight, c is protein concentration, R (θ) is the excess Rayleigh ratio, and K as the optical constant is described in equation 4:

$$K = \frac{4 {π}^{2} {{n}_{0}^{2} ( d n / d c )}^{2}}{{N}_{A} {λ}^{4}} \qquad ( Equation 4 )$$

wherein no is the solvent refractive index, do/dc denotes the increment of refractive index, N_{A} is Avogadro's number, and λ is the wavelength of the incident beam.

### 7.1 Differential Scanning Calorimetry Measurement

Differential scanning calorimetry (DSC), for example, MicroCal VP-DSC (Malvern Instruments, Worcestershire, UK), was employed to measure the apparent melting temperature (Tₘ) of proteins during thermal ramping. In a typical experiment setting, placebo and 1 mg/mL protein solutions at various formulation conditions were heated at 1° C./min from 20° C. to 105° C. Acquired thermosgram data were subtracted from placebo and analyzed for Tₘ with Origin 7.0 software using non-two state unfolding model.

### 8.1 Measurements of Agitation Stability

Surfactant free protein solution at 1 mg/mL was vortex mixed at 1000 rpm at room temperature. Subsequently, aggregation profiles were characterized by micro-flow imaging (MFI) and size exclusion chromatography (SEC).

### Example 1. Determinations of Physico-Chemical Properties of Bispecific Antibody and its Parental Antibodies

Protein homology modeling was conducted for BsAb1, mAb-A, and mAb-B, including sequence and structural analysis. Surface properties of the proteins were analyzed. The physico-chemical properties of bispecific antibody, for example, BsAb1, and its parental antibodies, for example, mAb-A and mAb-B, were determined as shown in Table 2. Based on the static model structures, the theoretical isoelectric points (pI) of the antibodies were determined. Experimental values of pI were measured using iCIEF. The experimental values of pI were similar to theoretical values of pI with slight differences. Interestingly, the pI values of BsAb1 is greater than that of mAb A, but less than that of mAb-B. Relative surface hydrophobicity measured by HIC-HPLC showed that BsAb1 and mAb-B had relatively higher hydrophobicity. Considering the similarities in the constant regions of mAb-A, mAb-B and BsAb1, the variable regions (Fv) of these antibodies were modelled to pinpoint the pI differences among them by determining Fv charge, Fv hydrophobicity index and Fv charge heterogeneity.

TABLE 2
Physico-chemical properties of BsAb1, mAb-A, and mAb-B.

| Physico-chemical | | | |
|---|---|---|---|
| Properties | BsAb1 | mAb-A | mAb-B |
| pI (theoretical) | 6.84 | 6.3 | 7.77 |
| pI (experimental, iCIEF) | About 6.9 | About 6.3 | About 7.3 |
| HΦ % (HIC-HPLC) | 30 | 20 | 32 |
| Fv surface charge | 3 | −1 | 8 |
| Fv hydrophobicity index | 1.15 | 1.08 | 1.23 |
| Fv charge heterogeneity | −13 | −53 | 9 |

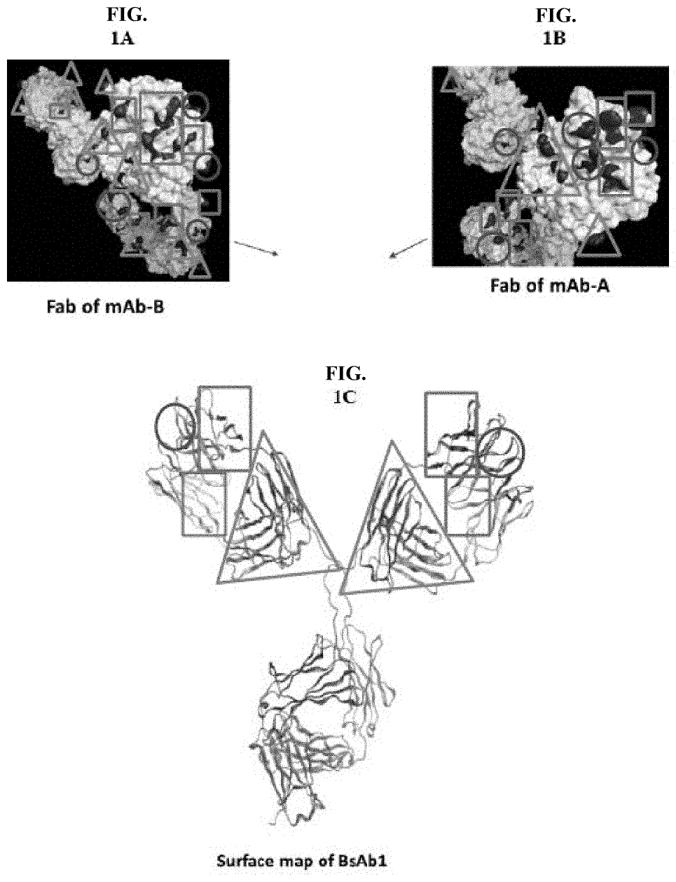

According to surface property analysis, the modeling results indicate that mAb-B has the highest Fv surface charge (total charge) and Fv hydrophobicity. The values of Fv surface charge and Fv hydrophobicity is in the order of mAb-B, BsAb1, and mAb-A from high to low. However, the value of Fv charge heterogeneity is in the reverse order as shown in Table 2. The structural modeling of BsAb1, Fab of mAb-A, and Fab of mAb-B as surface maps is shown in FIG. 1C, FIG. 1B and FIG. 1A, respectively. The shaded areas in the rectangles indicate the locations of hydrophobic patches. The shaded areas in the circles indicate the locations of negative charge patches. The shaded areas in the triangles indicate positive charge patches. As shown in the surface map in FIG. 1A and FIG. 1B, the two Fab arms of BsAb1 have distinctive surface properties, such as distinct surface charge and hydrophobicity. These results indicate that the high charge heterogeneity and low surface charge in the Fv region of mAb-A might attribute to adverse behaviors of mAb-A and BsAb1 at higher concentration.

The melting temperatures, e.g. Tₘ, of these antibodies were determined. The results indicate that there were no significant differences among melting temperatures. It suggests that the bispecific antibody, for example, BsAb1, and its parental antibodies, for example, mAb-A and mAb-B, have comparable conformational stability.

### Example 2. Measurements of Opalescence of Protein Formulations

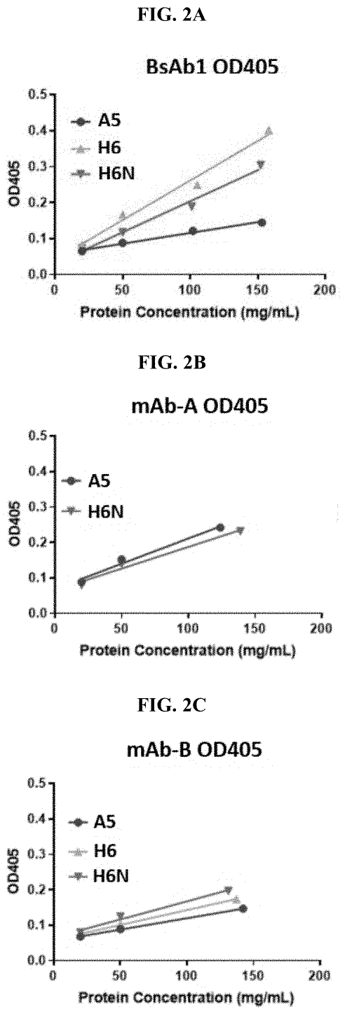

The opalescence of protein solutions was characterized by measuring optical density at 405 nm. The measurements of optical density at OD 405 nm were conducted for the protein formulations of BsAb1, mAb-A, and mAb-B as shown in FIG. 2A-2C. As shown in the target formulation buffers in Table 1, A5 indicates the composition of 10 mM acetate at pH 5. H6 indicates the composition of 10 mM histidine at pH 6. H6N indicates the composition of 10 mM histidine, 150 mM NaCl at pH 6. T8 indicates the composition of 10 mM Tris, at pH 8. When BsAb1 was prepared in the formulation buffer of 10 mM histidine at pH 6 (designated H6 in FIG. 2A), the protein concentrations of BsAb1 and the measurements of OD405 have linear dependences in the range of from 20 mg/mL to 150 mg/mL as shown in FIG. 2A. However, when the protein concentration of BsAb1 was above 50 mg/mL, significant haziness was visually observed. When 150 mM NaCl was included in the formulation buffer of BsAb1, opalescence was significantly reduced at protein concentrations above 50 mg/mL, and the measurements of OD450 are still linearly dependent on protein concentration of BsAb1. The effect of pH range was also investigated by preparing BsAb1 in the formulation buffer containing 10 mM acetate at pH 5 (designated A5 in FIG. 2A). Opalescence was significantly reduced, when pH value of the formulation buffer of BsAb1 was decreased, e.g. from pH 6 to pH 5. Conversely, there was a significant linear increase in Opalescence when pH was raised to 8. Although it is not shown in FIG. 2A, the Opalescence of BsAb1 in the formulation buffer containing 10 mM at pH 8 (designated as T8) was measured as show in Table 3:

TABLE 3
Opalescence of BsAb1 in Tris pH 8

| Concentration of BsAb1 (prepared in T8) | OD@405 |
|---|---|
| 20.4 mg/mL | 0.078 |
| 49.0 mg/mL | 0.125 |
| 67.2 mg/mL | 0.152 |

In contrast, parental mAb-A and mAb-B have completed different opalescence profiles in comparing to BsAb1 as shown in FIGS. 2B and 2C. mAb-A was unstable in formulation buffer of 10 mM histidine at pH 6, which suffered from severe precipitation and eventually underwent phase separation with the upper phase at 6.3 mg/mL protein concentration. When 150 mM NaCl was included in the formulation buffer of mAb-A to increase ionic strength, opalescence was significantly reduced and the solubility was increased (H6N in FIG. 2B). The effect of pH range was also investigated by preparing mAb-A in the formulation buffer containing 10 mM acetate at pH 5 (A5 in FIG. 2B). Opalescence was significantly reduced, when pH value of the formulation buffer of mAb-A was decreased, for example, from pH 6 to pH 5. mAb-B were prepared in three formulation buffers (FIG. 2C), and the measurements of OD405 of mAb-B were low in all three conditions. The strategy of reducing pH value (A5 in FIG. 2C) only made minimal impact, for example, from pH 6 to pH 5. When 150 mM NaCl was included in the formulation buffer of mAb-B to increase ionic strength, opalescence was slightly increased (H6N in FIG. 2C).

### Example 3. Measurements of Viscosities of Protein Formulations

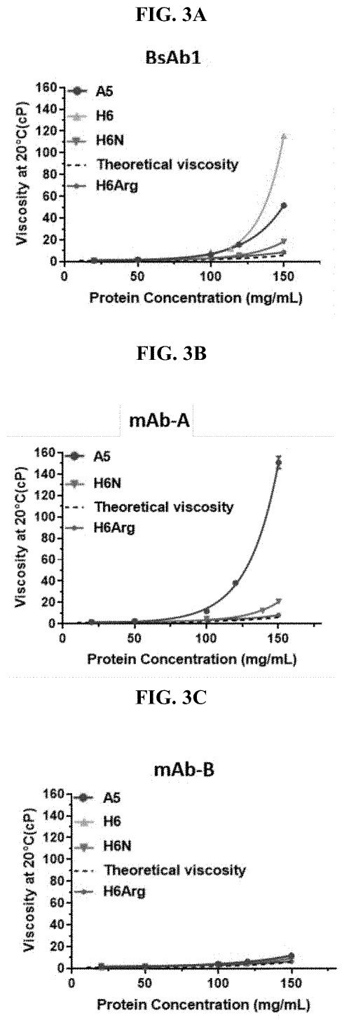

The solution viscosities of protein formulations at various conditions were measured using microchip-based viscometer. This approach can avoid the interference from air-water interface, and also take advantages of the feature of using small volumes of samples. BsAb1, mAb-A, or mAb-B were prepared in various formulation buffers as shown in FIG. 3A-3C, including the composition of 10 mM acetate at pH 5 (A5), the composition of 10 mM histidine at pH 6 (H6), the composition of 10 mM histidine, 150 mM NaCl at pH 6 (H6N), the composition of 10 mM histidine, 150 mM ArgHCl at pH 6 (H6Arg), and 10 mM Tris at pH 8 (T8). The viscosity of BsAb1 in 10 mM histidine, pH 6 showed an exponential dependence on protein concentration and reached as high as 120 cP at 150 mg/mL which is well exceeding the acceptable range for drug manufacturing and administration (H6 in FIG. 3A). As a comparison, theoretical viscosity of immunoglobulin with 10 nm diameter at 150 mg/mL was calculated by Mooney equation. The obtained results is only about 4 cP leading to the assumption that only hard sphere exclusion contributes to intermolecular interaction (FIG. 3A). The viscosities of the BsAb1 formulations were drastically reduced by either increasing ionic strength with the addition of 150 mM NaCl or by reducing pH value, e.g. from pH 8 to pH 5. When 150 mM ArgHCl was included in BsAb1 formulation (H6Arg in FIG. 3A), the viscosity of the BsAb1 formulation was reduced to the greatest extend among all formulation buffers. The viscosities of the BsAb1 formulation containing 150 mM ArgHCl across various protein concentrations were similar to the theoretical viscosities calculated and predicted by Mooney equation. Changes in ionic strength and pH values showed similar effects for mAb-A formulations (FIG. 3B). Although not shown in FIG. 3A, the viscosities of BsAb1 in formulation T8 were also measured as shown in Table 4,

TABLE 4
Viscosity of BsAb1 in Tris pH 8

| Concentration of BsAb1 (prepared in T8) | Viscosity (cp) |
|---|---|
| 20.4 mg/mL | 1.27 |
| 49.0 mg/mL | 1.85 |
| 67.2 mg/mL | 2.37 |

As discussed in the previous example (FIG. 2B), mAb-A was able to be re-solubilized at above 150 mg/mL by adding 150 mM NaCl or reducing pH, for example, from pH 6 to pH 5. However, at the protein concentration of 150 mg/mL, the viscosity of mAb-A in 10 mM acetate at pH 5 (A5) was significantly higher than that in 10 mM histidine, 150 mM NaCl, at pH 6 (H6N) as shown in FIG. 3B. The use of 150 mM ArgHCl was also able to solubilize mAb-A up to 150 mg/mL (H6Arg in FIG. 3B). In addition, the use of 150 mM ArgHCl in mAb-A formulation further reduced the viscosities to close to theoretically predicted values. In contrast, the viscosities of mAb-B formulations at various pH values or ionic strength are similar and comparable. The use of 150 mM ArgHCl in mAb-B formulation does not contribute to significant differences, which only slightly improved the viscosity to theoretical values (FIG. 3C).

Since the addition of ArgHCl can significantly reduce the viscosities of the BsAb1, mAb-A and mAb-B formulations, it indicates that the behaviors of these proteins at high concentration were influenced by surface hydrophobicity of these proteins. It is likely that minimal cross-interactions exist between the two heterogeneous Fab arms of BsAb1. The high viscosity and opalescence of BsAb1 is likely due to the Fab arm from mAb-A.

### Example 4. Effect of Ionic Strength, pH and Excipient in Agitation Stability

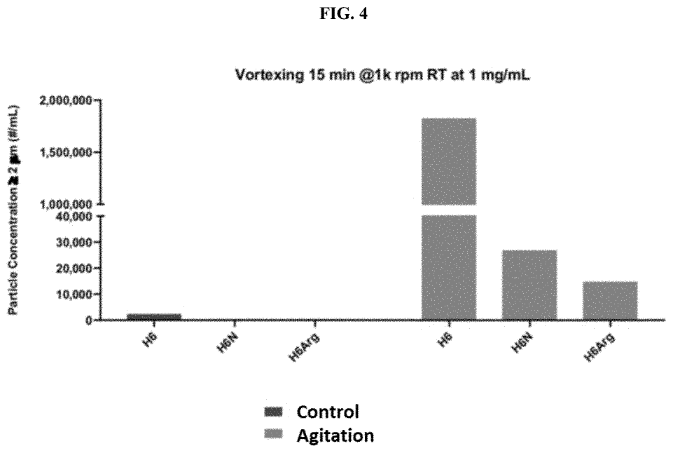

Agitation stability was measured to investigate the interfacial sensitivity of BsAb1 in various formulations. Protein solution at 1 mg/mL was vortex mixed at 1000 rpm at room temperature. Subsequently, aggregation profiles were characterized by micro-flow imaging (MFI) and size exclusion chromatography (SEC). As shown in FIG. 4, the strategies of increasing ionic strength can significantly reduce the formation of sub-visible particle upon agitation. The presence of ArgHCl can provide improvement of air-water interfacial stability. The results indicate that the interfacial sensitivity of BsAb1 is significantly modulated by protein-protein interaction. As shown in FIG. 4, H6 indicates the composition of 10 mM histidine at pH 6. H6N indicates the composition of 10 mM histidine, 150 mM NaCl at pH 6. H6Arg indicates the buffer composition of 10 mM histidine, 150 mM ArgHCl at pH 6.

### Example 5. Measurements of Interaction Parameter and Second Osmotic Coefficient

Interaction parameter k_{D} was measured using dynamic light scattering (DLS), such as Wyatt DynaPro plate reader (Wyatt Technology, CA) from 2 mg/mL to 10 mg/mL. The k_{D} value was estimated as described in the method section. Second osmotic viral coefficient B₂₂ was measured by Wyatt composition gradient-multi-angle light scattering (CG-MALS) system as described in the method section.

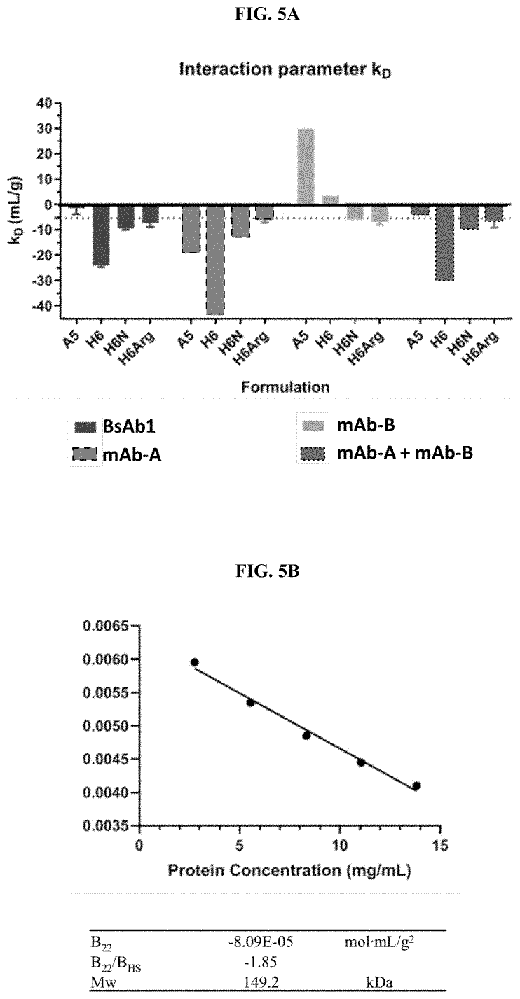

Protein interaction parameters (k_{D}) were measured by DLS. The results show that the interaction parameter k_{D} of BsAb1 has a significant negative value in the presence of 10 mM histidine at pH 6 (H6 in FIG. 5A) and pH 8 (Table 5), indicating the presence of strong attractive protein-protein interactions at pH 6. The strategies of increasing ionic strength (adding 150 mM NaCl or 150 mM ArgHCl) and changing pH value (from pH 6 to pH 5) can increase the k_{D} values of the BsAb1 formulations as shown in FIG. 5A. Although not shown in FIG. 5A, the viscosities of BsAb1 in additional formulations were also measured as shown in Table 5.

TABLE 5
Viscosity of BsAb1 in Tris pH 8

| Formulation Component of BsAb1 | k_{D} (mL/g) |
|---|---|
| Acetate, pH5.0 + Proline | −2.86 |
| Acetate, pH5.0 + Citrate | −12.5 |
| Acetate, pH5.0 + MgCl2 | −15.6 |
| Acetate, pH5.0 + Na2SO4 | −20.4 |
| His, pH6.0 + Proline | −23.4 |
| His, pH6.0 + Citrate | −11.7 |
| His, pH6.0 + MgCl2 | −11.9 |
| His, pH6.0 + Na2SO4 | −15 |
| Tris, pH8.0 | −7.03 |
| Tris, pH8.0 + Proline | −9.19 |
| Tris, pH8.0 + Citrate | −7.79 |
| Tris, pH8.0 + MgCl2 | −10.8 |
| Tris, pH8.0 + Na2SO4 | −8.3 |

As shown in FIG. 5A and Table 5, adding 150 mM ArgHCl or reducing pH value from pH 8 to pH 5 in BsAb1 formulations can increase k_{D} values of the BsAb1 formulations to theta condition where k_{D} is about −5.37 mL/g. When k_{D} value reaches theta condition, net protein-protein interaction does not exist except hard sphere repulsion at crowed concentrations.

Comparing with BsAb1, the interaction parameter k_{D} of mAb-A has a much larger negative value in the presence of 10 mM histidine at pH 6 (FIG. 5A), indicating the presence of much stronger attractive protein-protein interactions. The strategies of increasing ionic strength (adding 150 mM NaCl or 150 mM ArgHCl) and changing pH value (from pH 8 to pH 5) can increase the k_{D} values of the mAb-A formulations as shown in FIG. 5A and Table 5. However, the adjusted k_{D} values of mAb-A formulations are still more negative compared with those of BsAb1 formulations in corresponding formulations. As shown in FIG. 5A and Table 5, adding 150 mM ArgHCl in mAb-A formulations can increase k_{D} values of the mAb-A formulations to theta condition. In contrast, the k_{D} values of mAb-B formulations were close to or above theta condition as shown in FIG. 5A (error=std-dev of triplicates). Co-formulation of mAb-A and mAb-B have similar level of protein-protein interactions in comparing to BsAb1. Based on the results, increasing ionic strength, lowering pH value, and using hydrophobic excipient can effectively reduce attractive protein-protein interaction.

The intermolecular interaction parameter k_{D} analysis showed that significant attractive protein-protein interactions are present among BsAb1 molecules in 10 mM histidine at pH 6. The pH value of the formulation at pH 6 is close to BsAb1's pI of about 6.9. Therefore, it has been demonstrated that the strategy of adding 150 mM NaCl to the formulation can significantly reduce protein-protein interaction to a minimal level. Considering the overall low protein charge of BsAb1 at pH 6, since the adjustment of ionic strength can introduce significant effects to reduce viscosity of the formulation, it suggests that short range electrostatic interactions, such as dipole-dipole interaction, are responsible for the attractive protein-protein interactions which may contribute to the adverse high concentration behaviors of BsAb1.

High viscosity and opalescence of BsAb1 at high protein concentration were attributed by short range electrostatic interaction and hydrophobic interaction, with one Fab arm dominating the attractive self-interaction. Therefore, adverse high concentration behaviors of BsAb1 in the formulation can be effectively mitigated by increasing ionic strength and/or adding hydrophobic excipients. These results demonstrate that intermolecular interaction parameter k_{D} of BsAb1 can provide reasonable prediction for protein high concentration behaviors. Therefore, the measurements of k_{D} can be used to select molecular candidates for constructing bispecific antibodies and formulation optimization thereof.

Second osmotic viral coefficient B₂₂ of BsAb1 formulation in 10 mM histidine at pH 6 at various protein concentrations was measured by Wyatt composition gradient-multi-angle light scattering (CG-MALS) as shown in FIG. 5B. The results show large negative B₂₂ values which indicate the presence of strong attractive protein-protein interaction in the BsAb1 formulation in the presence of 10 mM histidine at pH6. These results confirmed that the prediction of protein-protein interaction was reliable based on the measurement of interaction parameter k_{D}.

### Example 6. Correlation Analysis Associated with Interaction Parameters

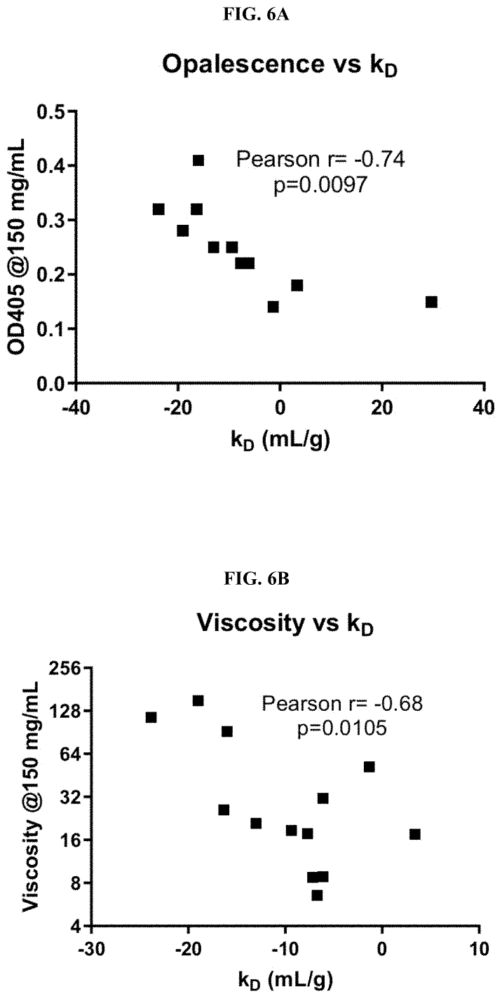

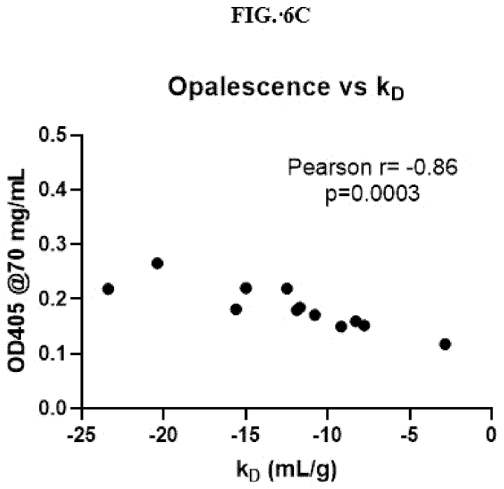

The relationship between opalescence and interaction parameter k_{D} were analyzed using Pearson correlation as shown in FIG. 6A (at a concentration of 150 mg/mL) and FIG. 6C (at a concentration of 70 mg/mL). Although not shown in FIG. 6A, the relationship between opalescence and interaction parameter k_{D} of BsAb1 in additional formulations were also measured as shown in Table 6.

TABLE 5
Viscosity of BsAb1 in Tris pH 8

| Formulation Component of BsAb1 | OD405 (at 70 mg/mL) |
|---|---|
| Acetate, pH5.0 + Proline | 0.1179 |
| Acetate, pH5.0 + Citrate | 0.2194 |
| Acetate, pH5.0 + MgCl2 | 0.1815 |
| Acetate, pH5.0 + Na2SO4 | 0.2657 |
| His, pH6.0 + Proline | 0.2187 |
| His, pH6.0 + Citrate | 0.1848 |
| His, pH6.0 + MgCl2 | 0.1799 |
| His, pH6.0 + Na2SO4 | 0.2202 |
| Tris, pH8.0 | 0.152 (at 67 mg/mL) |
| Tris, pH8.0 + Proline | 0.15 |
| Tris, pH8.0 + Citrate | 0.152 |
| Tris, pH8.0 + MgCl2 | 0.1711 |
| Tris, pH8.0 + Na2SO4 | 0.1595 |

The relationship between viscosity and interaction parameter k_{D} were analyzed using Pearson correlation as shown in FIG. 6B. The Pearson correlation coefficient, for example, Pearson r, is a measurement of the linear correlation between two variables. Reasonable correlation exists between opalescence/viscosity and interaction parameter k_{D}. The results indicate the existence of reasonable correlation between opalescence/viscosity and protein-protein interactions. The protein-protein interactions dominate the characteristics of proteins (protein behavior) at high protein concentration in solution.

What is claimed is:

1. A method for producing a combination of peptides or proteins with target physico-chemical properties, comprising:
receiving a plurality of amino acid sequences of the peptides or proteins,
selecting the peptides or proteins having desired amino acid sequences,
determining a profile of protein-protein interactions of the peptides or proteins having desired amino acid sequences,
selecting a target profile of the protein-protein interactions of the peptides or proteins having desired amino acid sequences, and
producing a combination of peptides or proteins having desired amino acid sequences according to the target profile of the protein-protein interactions.

2. The method of claim 1, wherein the profile of the protein-protein interactions is determined by measuring interaction parameters of the peptides or proteins having desired amino acid sequences.

3. The method of claim 1 further comprising determining a profile of physico-chemical properties of the peptides or proteins having desired amino acid sequences, wherein the combination of peptides or proteins having desired amino acid sequences is produced according to the target profile of the protein-protein interaction and the profile of the physico-chemical properties.

4. The method of claim 3, wherein the physico-chemical property is a theoretical isoelectric point, an experimental isoelectric point, surface hydrophobicity, relative surface hydrophobicity, a hydrophobicity index, surface charges, charge heterogeneity, second osmotic vial coefficient, agitation stability, opalescence, viscosity, or interfacial sensitivity.

5. The method of claim 4, wherein the surface hydrophobicity or surface charges is determined by conducting a structural modeling of the peptides or proteins having desired amino acid sequences.

6. The method of claim 1, wherein the protein-protein interactions are repulsive or attractive protein-protein interactions.

7. The method of claim 2, wherein the combination of the peptides or proteins having desired amino acid sequences is a bispecific antibody or a multi-specific antibody.

8. The method of claim 7, further comprising determining a hydrophobicity index, surface charges, or charge heterogeneity of a variable region of the bispecific antibody or the multi-specific antibody for producing the bispecific antibody or the multi-specific antibody.

9. The method of claim 1, wherein a concentration of the combination of the peptides or proteins having desired amino acid sequences is from about 20 mg/mL to about 200 mg/mL.

10. The method of claim 1, wherein a concentration of the combination of the peptides or proteins having desired amino acid sequences is at least about 70 mg/mL.

11. A system for producing a combination of peptides or proteins with target physico-chemical properties, comprising:
a first data storage including a plurality of amino acid sequences of the peptides or proteins,
a first processor coupled to the first data storage capable of making a selection of the peptides or proteins having desired amino acid sequences, and
a second processor capable of generating a profile of protein-protein interactions of the peptides or proteins having desired amino acid sequences, selecting a target profile of the protein-protein interactions of the peptides or proteins having desired amino acid sequences, and identifying a combination of peptides or proteins having desired amino acid sequences, wherein the combination of the peptides or proteins having desired amino acid sequences are selected according to the target profile of the protein-protein interactions.

12. The system of claim 11, wherein the profile of the protein-protein interactions is determined by measuring interaction parameters of the peptides or proteins having desired amino acid sequences.

13. The system of claim 11, further comprising a profile of physico-chemical properties of the peptides or proteins having desired amino acid sequences, wherein the combination of peptides or proteins having desired amino acid sequences is selected according to the target profile of the protein-protein interaction and the profile of the physico-chemical properties.

14. The system of claim 13, wherein the physico-chemical property is a theoretical isoelectric point, an experimental isoelectric point, surface hydrophobicity, relative surface hydrophobicity, a hydrophobicity index, surface charges, charge heterogeneity, second osmotic vial coefficient, agitation stability, opalescence, viscosity, or interfacial sensitivity.

15. The system of claim 14, wherein the surface hydrophobicity or surface charges is determined by conducting a structural modeling of the peptides or proteins having desired amino acid sequences.

16. The system of claim 11, wherein the protein-protein interactions are repulsive or attractive protein-protein interactions.

17. The system of claim 11, wherein the combination of the peptides or proteins having desired amino acid sequences is a bispecific antibody or a multi-specific antibody.

18. The system of claim 17 further comprising a profile of a hydrophobicity index, surface charges, or charge heterogeneity of a variable region of the bispecific antibody or the multi-specific antibody.

19. The system of claim 11, wherein a concentration of the combination of the peptides or proteins having desired amino acid sequences is from about 20 mg/mL to about 200 mg/mL.

20. The system of claim 11, wherein a concentration of the combination of the peptides or proteins having desired amino acid sequences is at least about 70 mg/mL.

21. A method for optimizing or selecting at least one component in a formulation, wherein the formulation comprises the combination of the peptides or proteins having desired amino acid sequences of claim 1, the method comprising:
adjusting ionic strength of the formulation based on the target profile of the protein-protein interactions of the peptides or proteins having desired amino acid sequences, and
adjusting a pH value of the formulation based on the target profile of the protein-protein interactions of the peptides or proteins having desired amino acid sequences.

22. The method of claim 21 further comprising adding a salt to the formulation based on the target profile of the protein-protein interactions of the peptides or proteins having desired amino acid sequences.

23. The method of claim 21 further comprising adding a hydrophobic excipient to the formulation based on the target profile of the protein-protein interactions of the peptides or proteins having desired amino acid sequences.

24. The method of claim 21, wherein the at least one component is sodium chloride, acetate, histidine, or arginine hydrochloride.

25. A method for optimizing or selecting at least one component in a formulation, wherein the formulation comprises a bispecific antibody or a multi-specific antibody, the method comprising:
determining a profile of protein-protein interactions of the bispecific antibody or the multi-specific antibody, and
optimizing or selecting the at least one component in the formulation based on the profile of the protein-protein interactions of the bispecific antibody or the multi-specific antibody.

26. The method of claim 25, wherein the profile of the protein-protein interactions is determined by measuring interaction parameters of the bispecific antibody or the multi-specific antibody.

27. The method of claim 25, further comprising, adjusting ionic strength of the formulation based on the profile of the protein-protein interactions of the bispecific antibody or the multi-specific antibody.

28. The method of claim 25, further comprising, adjusting a pH value of the formulation based on the profile of the protein-protein interactions of the bispecific antibody or the multi-specific antibody.

29. The method of claim 25, further comprising determining a profile of physico-chemical properties of the bispecific antibody or the multi-specific antibody, wherein optimizing or selecting the at least one component in the formulation is based on the profile of the protein-protein interactions of the bispecific antibody or the multi-specific antibody and the profile of the physico-chemical properties of the bispecific antibody or the multi-specific antibody.

30. The method of claim 25, further comprising adding a salt to the formulation based on the profile of protein-protein interactions of the bispecific antibody or the multi-specific antibody.

31. The method of claim 25, further comprising adding a hydrophobic excipient to the formulation based on the profile of protein-protein interactions of the bispecific antibody or the multi-specific antibody.

32. The method of claim 25, wherein at least one component is sodium chloride, acetate, histidine, or arginine hydrochloride.

33. The method of claim 29, wherein the physico-chemical property is a theoretical isoelectric point, an experimental isoelectric point, surface hydrophobicity, relative surface hydrophobicity, a hydrophobicity index, surface charges, charge heterogeneity, second osmotic vial coefficient, agitation stability, opalescence, viscosity, or interfacial sensitivity.

34. The method of claim 33, wherein the surface hydrophobicity or surface charges is determined by conducting a structural modeling of the bispecific antibody or the multi-specific antibody.

35. The method of claim 25, wherein the protein-protein interactions are repulsive or attractive protein-protein interactions.

36. The method of claim 29, further comprising determining a hydrophobicity index, surface charges, or charge heterogeneity of a variable region of the bispecific antibody or the multi-specific antibody.

37. The method of claim 25, wherein a concentration of the bispecific antibody or the multi-specific antibody is from about 20 mg/mL to about 200 mg/mL.

38. The method of claim 25, wherein a concentration of the bispecific antibody or the multi-specific antibody is at least about 70 mg/mL.
